(19) United States
(12) Patent Application Publication
(10) Pub. No.: US 2011/0224624 A1
(43) Pub. Date: Sep. 15, 2011

(54) VALVE ON A CATHETER-LIKE OR A CHARGING VALVE-LIKE MEDICAL INSTALLATION

(76) Inventor: Johan-Christoph Geller, Weimar (DE)

(21) Appl. No.: 13/016,268

(22) Filed: Jan. 28, 2011

(30) Foreign Application Priority Data
Jul. 29, 2009 (DE) 10 2009 035 256.2

(51) Int. Cl. A61M 25/14

(52) U.S. Cl. 604/246

(57) ABSTRACT

The invention relates to a valve, which is fixedly connected to a catheter-like or charging valve-like medical installation for supplying drugs to a patient through a blood vessel or for withdrawing a body fluid from a blood vessel, with a blocking device being provided on the valve, ensuring trouble-free retraction of the medical installation from the vessel or replacement of the medical installation. This is preferably a three-way valve.

The invention relates to a valve having a deflectable valve lever. The valve is disposed on the free end of an intravascular catheter-like or charging valve-like medical installation for supplying pharmaceutical drugs to a human or animal blood vessel or for controllable withdrawal of body fluids from a human or animal vessel, for example, in tests on the heart.

The use of three-way valves as proximal end parts of catheters or charging valves is known in medical technology, for example. When an intravascular introducer charging valve is connected to a patient's blood vessel at one of the three connections of a three-way valve, for example, one drug may be dispensed into the introducer charging valve into the vessel optionally by way of the second or third connection, depending on the position of the valve lever, or two different drugs may be dispensed into the introducer charging valve at the same time.

In the case of a cardiac catheter examination, for example, additional catheters and charging valves may optionally be needed in the immediate vicinity in addition to the introducer charging valve.

In the prior art there is often the problem that in the valve cutoff position of the three-way valve to the introducer charging valve, in which the valve lever usually points in the direction of the introducer charging valve and the introducer charging valve can be retracted out of the vessel or replaced, the control lever of the three-way valve may easily become snagged or jammed with the additional catheters and charging valves being used in a cardiac catheter examination, for example, which can lead to substantial complications that could substantially endanger or impair the treatment for example.

The object of the invention is therefore to offer a solution to the problem, which will ensure a trouble-free retraction or replacement of an introducer charging valve, for example, or a catheter in the cutoff position to the three-way valve, in which the valve lever points in the direction of the introducer charging valve in the usual manner. The attending physician can sense this position even in weak lighting to ensure, for example, that before retraction or replacement of the introducer charging valve, the three-way valve is in its closing position with respect to the introducer charging valve. When the valve lever is pivoted into this closing position, the valve lever can easily become snagged or jammed with the other catheters or charging valves that are additionally present in the immediate vicinity before reaching the fully closed position, so that then it is difficult or impossible to retract the introducer charging valve out of the vessel or to replace the introducer charging valve by removing it from the vessel.

The problem on which the invention is based is solved according to the invention through the features of Claim 1. Advantageous embodiments of the inventive object described in Claim 1 can be derived from the subsidiary claims and the following description.

The invention is not limited to the features in the claims. On the basis of these features, alternative approaches that are readily self-evident to the skilled person in the art, all of which are within the scope of the general inventive idea, are suggested here.

The invention will now be described and explained without restriction on the basis of the following drawings, in which:

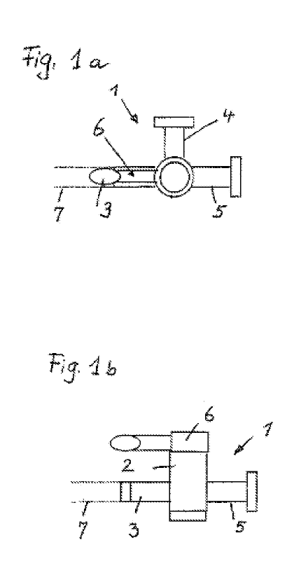

FIG. 1a shows a conventional three-way valve, for example, in combination with an introducer charging valve in a view from above;

FIG. 1b shows the conventional three-way valve according to FIG. 1a in a side view;

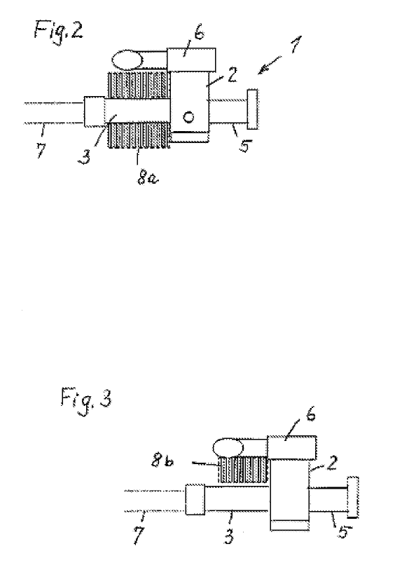

FIG. 2 shows a three-way valve according to the invention in a first embodiment in a side view;

FIG. 3 shows a three-way valve according to the invention in a second embodiment in a side view;

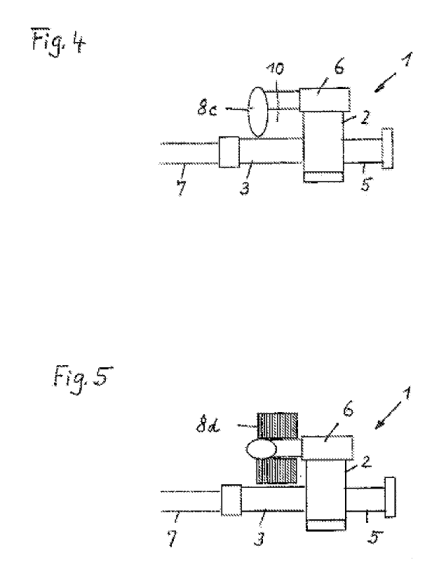

FIG. 4 shows a three-way valve according to the invention in a third embodiment in a side view;

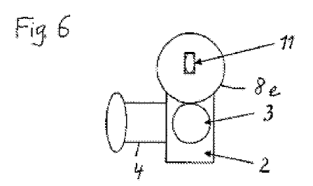

FIG. 5 shows a three-way valve according to the invention in a fourth embodiment in a side view;

FIG. 6 shows a three-way valve according to the invention in a fifth embodiment in the view from the front of the connection of the valve to the connection to the introducer charging valve (not shown).

FIGS. 1a and 1b show a traditional three-way valve, labeled as 1, in a view from above (FIG. 1a) and in a side view (FIG. 1b). The cylindrical valve housing is labeled as 2, with three radial connections 3, 4 and 5 leading away from the valve housing, each connection forming a 90° angle to the others. A known cylindrical valve body having a T-shaped control channel is rotatably mounted in the cylindrical valve housing (not shown in detail here), where the valve body protrudes slightly beyond the upper end of the valve housing 2 axially, to which a valve lever 6 of a predetermined length extending radially outward is connected in a rotationally fixed manner for rotating the valve body.

FIG. 1a shows the pivotable valve lever 6 radially in the direction of the introducer charging valves (not shown further) which are used here, for example, and are fixedly connected to the valve connection end 7 at the one lengthened connection 3 of the three-way valve 1.

A supply line for drugs (not shown) is detachably connected to each of the two other connections 4 and 5.

By rotating the valve lever 6 counterclockwise by 90° out of its closed position shown in FIGS. 1a and 1b in the direction of the connection 4 with respect to the introducer charging valve 7, the connection 5 is cut off and at the same time this connection is connected to the introducer charging valve 7 through the open connection 3.

By further rotating the valve lever 6 clockwise by 90°, the valve lever 6 points in the direction of the connection 5, in which the valve is cut off and instead the connection 4 is connected to the open connection 3 to the introducer charging valve 7.

By further rotating the valve lever 6 by 90° in a direction opposite the connection 4, both connections 4 and 5 are connected to the open connection 3.

Medication is dispensed into the introducer charging valve through connection 3 and/or 4, depending on which position of the valve lever 6 is selected.

If the valve lever 6 points into the gasket of one of the connections 3, 4 or 5, then, as stated previously, this shows the attending physician the closing position of the respective connection 3 and/or 4 and/or 5.

In FIGS. 2 through 6, which show five exemplary embodiments of the three-way valve according to the present invention without restriction, corresponding valve parts are labeled with the same reference numerals as those used in FIGS. 1a and 1b.

The devices on the inventive three-way valve for trouble-free retraction of the introducer charging valve 7 from the treated vessel or for replacing the introducer charging valve 7 are each labeled with reference numeral 8 as the basic numeral, differentiating them by indices a, b, c, d and e.

The blocking device in FIG. 2 consists of a ring body 8a, which is fitted to the diameter of the lengthened connection 3 and is pushed over the connection 3 of the three-way valve 1 as far as the stop on the valve housing 2. The ring body 8a has a radial diameter and an axial width, such that the free gap between the bottom side of the valve lever 6 and the top side of the connection 3 of the three-way valve 1 is mostly closed in such a way that the free pivotability of the valve lever is not hindered.

The ring-shaped blocking device 8a may be fixedly connected to the lengthened connection 3. However, it may also be replaceably pushed onto connection 3.

A securing sleeve 9, which may optionally also be omitted, is provided on the front end of the connection to prevent unintentional removal of the blocking device 8a from the connection 3.

The blocking device 8a may be made of any material which fulfills the required blocking function here, in particular plastic, which has proven suitable in comparable medical installations.

In FIG. 3 the blocking device 8b comprises a strip-shaped material, which is fixedly arranged here on the bottom side of the valve lever 6, for example, and fills up the valve lever 6 for sealing the connection 3, filling the free gap from the bottom side of the valve lever 6 up to tightly against the top side of the connection 3 in the closed position, such that the free pivotability of the valve lever is not restricted. The blocking device could also be arranged on the top side of the connection 3 accordingly.

In FIG. 4 the blocking device 8c is formed from a shield-like part, which is fixedly or detachably arranged on the end face of the valve lever 6 and protrudes only slightly beyond the upper end of the valve lever but extends with its lower end at a right angle to the valve lever 6 perpendicularly downward close to the outside surface of the connection 3. In this way the free gap 10 between the bottom side of the valve lever 6 and the top side of the connection 3 is closed securely from the outside to reliably prevent snagging or jamming of the valve lever 6 with the additional charging valves or catheters.

FIG. 5 shows another variant of the blocking device 8d, which is designed as a ring-shaped body in FIG. 5, comparable to the approach in FIG. 2, and is pushed at its central opening over a radial section of the valve lever 6 and is arranged here fixedly or removably on the valve lever 6. The ring body 8d here is at a small distance from the valve housing 2 to maintain an unhindered pivoting movement of the valve lever 6.

The blocking device according to FIG. 6 comprises a ring-shaped disk body 8e, which has a central opening 11 for attaching the disk-type blocking body 8e to the front end of the valve lever 6, so that, as in the approach according to FIG. 4, the free gap 10 between the bottom side of the valve lever 6 and the top side of the connection 3 is tightly closed off in a manner such that snagging or jamming with additional charging valves and/or catheters is reliably prevented.

Here again, the ring-shaped disk body 8e can be fixedly connected to the outer end of the valve lever 6 or may also be detachably attached to the front end of the valve lever.

The shield-like part 8c in FIG. 4 and the ring-shaped disk body 8e in FIG. 6, each one on the front end of the valve lever 6, may also be designed in one piece with the latter.

The variants described above for the blocking devices 8 are either fixedly or detachably arranged on the lengthened connection 3 for the introducer charging valve 7 or on the valve lever 6. Those skilled in the art in the respective field will readily obtain suggestions for additional variants within the scope of the present inventive idea from these embodiments of the invention for trouble-free handling, for example, an introducer charging valve connected to a three-way valve.

The blocking devices according to the invention ensure that on retraction of an introducer charging valve out of a blood vessel, for example, or on replacement of the introducer charging valve, the valve lever 6 of the three-way valve 1 is not snagged or jammed with additional catheters or introducer charging valves, so that retraction or replacement of the introducer charging valve is blocked or made difficult and is possible only after a time lag, after which the jamming or snagging with the other charging valves and catheters has been resolved.

1. A valve which is fixedly connected as the end part to a catheter-like or charging valve-like medical installation for supplying medicines into a vessel of a patient or for withdrawing body fluid from a vessel,
characterized in that
a blocking device (8) which ensures a trouble-free retraction of the medical installation out of the vessel or replacement of the medical installation is provided.

2. The valve according to claim 1,
characterized in that
the valve is a three-way valve (1).

3. The valve according to claim 2,
characterized in that
the blocking device (8) is arranged on the radially lengthened pipe connection (3) of the three-way valve (1) with a connection to the medical installation (8).

4. The valve according to claim 3,
characterized in that
the blocking device (8) is arranged on the control lever (6) of the three-way valve.

5. The valve according to claim 1,
characterized in that
the blocking device (8) claims the access from the outside into the free gap (10) between the control lever (6) and the lengthened pipe connection (3) with a connection to the medical installation.

6. The valve according to claim 5,
characterized in that
the blocking device (8) blocks access from the outside to the free gap (10) between the control lever (6) and the lengthened pipe connection (3) of the valve.

7. The valve according to claim 6,
characterized in that
the blocking device (8) is arranged on the front end of the valve lever (6).

8. The valve according to claim 6,
characterized in that
the blocking device (8) is arranged on the lengthened pipe connection (3).

9. The valve according to claim 6,
characterized in that
the valve lever or the pipe connection (3) is designed in one piece with the blocking device (8).
